(19) United States
(12) Patent Application Publication
(10) Pub. No.: US 2004/0052773 A1
(43) Pub. Date: Mar. 18, 2004

(54) Production of multi-chain protein from muscle

(75) Inventors: Bjarne Bogen, Snaroya (NO); Iacob Mathiesen, Oslo (NO); Torunn Tjelle, Oslo (NO)

Correspondence Address: FOLEY AND LARDNER, SUITE 500, 3000 K STREET NW, WASHINGTON, DC, 20007, US

(73) Assignee: Inovio AS

(21) Appl. No.: 10/345,410

(22) Filed: Jan. 16, 2003

Related U.S. Application Data
(60) Provisional application No. 60/350,309, filed on Jan. 18, 2002.

(51) Int. Cl. A61K 48/00; A61K 39/395

(52) U.S. Cl. 424/93.21; 424/130.1

(57) ABSTRACT

A multi-chain protein can be produced in a subject by intramuscular injection of one or more vectors that code for the chains of the protein and, optionally, by applying one or more electrical pulses across the injection site. A preferred multi-chain protein is an immunoglobulin. This approach to antibody production has various applications, including for expressing multi-chain proteins in vivo for disease therapy and for eliciting an immune response to one or more foreign antigenic determinants of the expressed protein.

### BACKGROUND OF THE INVENTION

This invention relates to expressing multi-chain proteins from muscle in vivo.

A number of genetic changes have been identified to cause disease (e.g., cancer, muscular dystrophy and cystic fibrosis) and delivery of functional exogenous genes to cells (i.e., “genetic delivery”) has been proposed as a therapeutic strategy. Various approaches for genetic delivery have been considered, with some limited success. See Rosenberg et al. New Eng. J. Med. 323, 570 (1990).

Viral vectors have been widely used for genetic delivery due to the relatively high efficiency of transfection and potential for long term expression resulting from actual integration of the vector DNA into the host's genome. However, there are risks involved in the use of viruses, such as activation of proto-oncogenes, reversion to a wild-type virus from a replication incompetent virus, immunogenicity of viral proteins, and the adjuvant effect of viral proteins on the immunogenicity of the expressed transgene.

The discovery that naked DNA is taken up and transiently expressed by muscle cells in vivo has increased interest in using non-viral vehicles for genetic delivery. See Wolff et al., Science 247, 1465 (1990); Acsadi et al., Nature 352, 815 (1991). The mechanisms for cellular uptake of exogenous DNA and subsequent expression are not clear. Also, the efficiency of transfer is low, with only transient expression of up to a few weeks or a few months generally observed. Although genetic delivery is a promising area of research, new methods for introducing genes and achieving a useful level of gene product expression in vivo are needed.

### SUMMARY OF THE INVENTION

In accordance with one aspect of the present invention, a methodology is provided for producing a protein that comprises at least two different polypeptide chains in the circulation of an individual. The method comprises injecting into the muscle of the individual at least one expression vector encoding the polypeptide chains, such that uptake of the vector into muscle cells results in secretion of the protein. The DNA encoding each chain may be located on a single vector or on separate vectors. In a preferred embodiment, one or more electrical pulses is applied to the muscle at the site of injection to enhance expression of the protein.

In some embodiments, the protein comprises one or more antigenic determinants foreign to the individual, thereby generating an immune response to the expressed protein in the individual. The immune response in the individual can include the production of antibodies in the serum to the one or more foreign antigenic determinants of the protein.

In accordance with another aspect of the present invention, an approach is provided for obtaining antibodies to a protein that comprises at least two different polypeptide chains. The approach comprises injecting into muscle of an individual at least one expression vector that encodes the polypeptide chains, such that uptake of the vector into muscle cells results in secretion of the protein. In accordance with this approach, the protein comprises one or more antigenic determinants foreign to the individual which results in the production of antibodies by the individual. In furtherance of this approach, the antibodies are obtained from the individual, preferably from the circulation. The DNA encoding each chain may be located on a single vector or on separate vectors. In a preferred embodiment, one or more electrical pulses is applied to the muscle at the site of injection to enhance expression of the protein.

In accordance with a further aspect of the present invention, an immunizing procedure is provided that comprises injecting at least one expression vector encoding the light chains and the heavy chains of a bispecific antibody i.m. into muscle of an individual, the bispecific antibody having a first binding site specific for a cell surface marker of an antigen presenting cell of the individual and a second binding site specific for an antigen to which immunization is desired. Uptake of the vector into muscle cells of the individual following injection results in secretion of the bispecific antibody in the circulation of the individual. In furtherance of this procedure, antigen is administered to the individual and targeted to antigen presenting cells by the bispecific antibody. The DNA encoding each chain may be located on a single vector or on separate vectors. In a preferred embodiment, one or more electrical pulses is applied to the muscle at the site of injection to enhance expression of the protein.

In accordance with a yet a further aspect of the present invention, an immunizing procedure is provided that comprises injecting at least one expression vector into the muscle of the individual, the vector encoding an antibody fusion protein, the fusion protein comprising an antibody specific for a cell surface marker of an antigen presenting cell of the individual, the antibody fused to a polypeptide antigen to which immunization is desired, wherein uptake of the vector into muscle cells results in secretion of the antibody fusion protein. In accordance with this procedure, the secreted fusion protein functions to target the antigen to the surface of antigen presenting cells of the individual. The DNA encoding each chain may be located on a single vector or on separate vectors. In a preferred embodiment, one or more electrical pulses is applied to the muscle at the site of injection to enhance expression of the protein.

In accordance with still another aspect of the present invention, a methodology for testing at least one biological activity of a protein. The method comprises injecting into muscle of the individual at least one expression vector encoding the polypeptide chains, such that uptake of the vector into muscle results in secretion of the protein, and then testing a biological activity of the expressed protein. In one embodiment, the biological activity occurs in the individual. In another embodiment, the expressed protein is removed from the individual and then tested for activity. The DNA encoding each chain may be located on a single vector or on separate vectors. In a preferred embodiment, one or more electrical pulses is applied to the muscle at the site of injection to enhance expression of the protein.

### BRIEF DESCRIPTION OF THE DRAWINGS

FIG. 1 demonstrates expression of recombinant immunoglobulin in the serum of mice administered immunoglobulin expression vectors in accordance with the invention. FIG. 1A is a graph that depicts serum levels of an expressed chimeric I-E^{d} specific monoclonal antibody (mAb) at day 6 in mice (BALB/c) injected with the indicated antibody expression vectors in muscle followed with (+) or without (−) electroporation (EP) at the injection site. FIG. 1B depicts serum levels of an expressed chimeric I-E^{d} specific antibody at the days indicated in mice (BALB/c and C57BL/6) injected with the combi expression vector (encoding heavy and light chain) in muscle followed with (+) or without (−) electroporation (EP) at the injection site. FIG. 1C depicts serum levels of an expressed chimeric I-E^{d} specific antibody at the days indicated in mice (BALB/c, Balb.B, B10.D2 and C57BL/6) injected with the combi expression vector in muscle followed with electroporation at the injection site. FIG. 1D depicts serum levels of an expressed chimeric IgD^{a} specific antibody at the days indicated in mice (BALB/c and C.B-17) co-administered heavy and light chain encoding expression vectors by i.m. muscle injection followed with electroporation at the injection site.

FIG. 2 demonstrates assembled antibody produced in accordance with the invention. Sera was obtained from mice co-administered heavy and light chain encoding expression vectors by i.m. muscle injection followed with electroporation at the injection site. Chimeric IgD^{a} specific antibody was concentrated from the sera by binding and elution from Protein G Sepharose beads. The eluate was treated or not-treated with mercaptoethanol (ME) prior to Western blotting with antibody specific for the light chain (anti-human kappa) and heavy chain (anti-human IgG3) of the expressed chimeric IgD^{a} specific antibody.

FIG. 3 demonstrates that anti-immunoglobulin antibodies are induced in mice expressing recombinant antibodies produced in accordance with the invention. Sera from animals in the experiments shown in FIGS. 1C and 1D for day 28 were analyzed in ELISA plates coated with human IgG3 immunoglobulin and detected using anti-mouse IgG1 (black bars) and IgG2a antibody (gray bars). The left hand panels represent sera from FIG. 1C while the right hand panel represents sera from FIG. 1D. Results are presented as antibody endpoint titer and error bars are standard error of the mean.

FIG. 4 illustrates serum mAb expression of mouse or chimeric antibody induced in accordance with the invention. FIG. 4A depicts the serum level of anti-IgD^{a} specific antibody in mice co-administered 50 μg of each of plasmids pLNOH2 [symbol] 2bVHT and pLNO [symbol] VLT (together encoding an anti-IgD^{a} mAb that has a complete mouse IgG2b heavy chain and a chimeric light chain (mouse variable domain, human Ckappa domain) into mice (BALB/c and C.B-17), followed with (+EP) or without (−EP) electroporation. Kinetics of serum mouse IgG2b with IgD^{a} specificity is shown. FIG. 4B depicts the serum level of 4-hydroxy-3-iodo-5-nitrophenylacetic acid (NIP)-specific antibody anti- in BALB/c mice co-administered 100 or 10 μg (main graph) or 50 μg (insert graph) of each of the plasmids pLNOH2γ2bVHNP and λ1, that together encode a fully mouse IgG2λ1 anti-NIP mAb, i.m., followed with EP or not followed by EP. Kinetics of serum mouse IgG2b with NIP specificity is shown. Each group consisted of 3-7 mice and the bars represent the standard error of mean.

FIG. 5 demonstrates that antibody expressed in the serum in accordance with the invention is biological active, has an intact Fc region, and normal glycosylation. Serum from mice that had been injected/electroporated with Ig genes encoding a mouse IgG2b anti-NIP mAb (open squares), or the corresponding mAb purified from supernatants of in vitro transfected cells (filled squares), were tested for the ability to lyse NIP sensitized ⁵¹Cr-labelled sheep red blood cells (SRBC) in the presence of human complement. As negative control, mouse IgG1 anti-NIP (clone N1-G9 mIgG1; filled diamonds) that does not activate complement was included. The cytotoxic index (CI) was calculated according to the formula: %CI=[(cpm test−cpm spontaneous)/(cpm max−cpm spontaneous)]×100.

FIG. 6 shows that antibody directed against a B lymphoid cell marker IgD can be expressed in the circulation in accordance with the method and result in depletion of IgD B cells in the individual. The percentage of IgD positive B cells is reduced in the group administered the vector and electroporated.

### DETAILED DESCRIPTION OF THE INVENTION

The invention methods are based on the discovery that muscle can support the expression of a multi-chain protein that is not normally expressed in muscle, and that such expression results in release and accumulation of detectable, active heteromultimeric protein in systemic circulation and/or absorbed to antigen expressed by tissues in the individual. Injection of plasmid DNA encoding the various polypeptides of the heteromultimer into skeletal muscle optimally combined with electroporation of the injection site, yields assembled heteromultimer with intact specificity and biological effector function. In vivo electroporation applied as low voltage, electrical pulses (one or more) so that the current passes through the DNA injection site is useful to expression of the heteromultimer in the circulation.

Accordingly, a method is provided for producing a protein in the circulation of an individual, the protein comprising at least two different polypeptide chains, the method comprising injecting into muscle of the individual at least one expression vector that encodes the polypeptide chains. In accordance with this method, uptake of the vector into muscle cells results in production of the polypeptide chains and secretion of the protein. In a preferred embodiment, one or more electrical pulses is applied to the muscle at the site of injection to enhance expression of the protein.

As used herein, the phrase “at least one expression vector that encodes the polypeptide chains” or “at least one expression vector that encodes the heavy and light chain” means that muscle is injected i.m. with a single vector that encodes all of the different chains of the multi-chain protein (or the heavy and light chains of an immunoglobulin) or is injected i.m. with separate vectors, each encoding one of the chains of the multi-chain protein. In the latter case, the separate vectors are preferably co-administered.

High and stable levels of muscle-produced heteromultimeric protein are possible using the invention methods. For example, IgG2b class mouse mAbs were produced in sera at a concentration of 400 ηg per ml for more than 7 months following a single bilateral muscle administration. The short serum half life of mouse IgG2b (about 4-5 days) indicates that mouse IgG2b is continuously produced by the muscle cells over the indicated time. This level of production may be further increased by various approaches discussed herein.

The invention methods for producing multi-chain protein from muscle provide an alternative to clinical scenarios where direct administration of the protein has therapeutic value. Thus, one may express a particular multi-chain protein that is active against a disease or condition in an individual suffering from that disease or condition, for the purposes of treating the individual. Such “therapeutic” multi-chain proteins are well known in the art and include, for example, antibodies, insulin and hemoglobin. For example, in the case of antibodies, expression from muscle as described herein may be an alternative or a supplement to passive antibody therapy for treatment of applicable diseases such as, for example, cancer and autoimmune disease including B lymphomas (anti-CD20 mAb, Colombat, et al. Blood 97, 101-106 (2001)), breast cancer (anti-Her 2; Leonard, et al. Br. J. Surg. 89, 262-271 (2002)) and rheumatoid arthritis (anti-TNFα, Feldmann, et al. Joint Bone Spine 69, 12-18 (2002)). Additional exemplary such antibodies are listed in Table 1, the encoding nucleotide sequences of which are available from public sequence repositories. Compared to passive administration of mAb, DNA injection/electroporation is less expensive, possesses less danger of infection, and may be applied as a single injection with long term effects and little to no side effects.

The invention methods can be applied to produce any of a variety of multi-chain proteins in the circulation of an individual. In a preferred embodiment, each of the chains of the multi-chain protein interact in such a manner as to form a ligand binding site or substrate binding site. Thus, multi-chain proteins may constitute any of a variety of heteromultimers such as heterodimers, heterotrimers, and the like.

Heteromultimer multi-chain proteins that can be expressed by the invention methods may also include multiple copies of a particular polypeptide. For example, as demonstrated herein, muscle can assemble immunoglobulin heavy (H) and light (L) chains as tetrameric (H+L)₂ molecules, even if separate plasmids for H- and L-chain genes are injected. Although not wishing to be bound by any theory, it is believed that a single muscle cell in vivo produces H- and L-chains which assemble as (H+L)₂ molecules prior to secretion. Importantly, the variable regions (V-regions) of muscle-produced monoclonal antibodies (mAbs) appear to have correctly because serum mAb produced in accordance with the invention exhibits the expected antigen specificity for its target antigen (e.g., NIP hapten, IgD or I-E^{d} class II MHC molecule). Also the Fc region of the expressed tetrameric (H+L)₂ molecules also appears appear to have correctly folded folded and glycosylated because muscle-produced mAb was able to activate complement. Since complement activation requires glycosylation (Tao et al. J. Immunol. 143, 2595-2601 (1989)), the result suggests that the muscle-produced Ig was suitably glycosylated.

In addition to expressing immunoglobulins, the present invention also may be used to express other heteromultimeric proteins including an MHC molecule, such as a class I or class II MHC molecule, in which two chains form a peptide binding pocket. A further example is a multi-chain enzyme that binds a substrate and catalyzes the formation of a product from the substrate. In other embodiments, a multi-chain protein that binds to a receptor at the cell surface, which will then lead to intracellular signaling, can be expressed in accordance with the present invention.

In these instances, the functional ligand binding site or substrate binding site is formed through stable association of the chains that is mediated through a variety of molecular forces including, for example, ionic, covalent, hydrophobic, van der Waals, and hydrogen bonding. Expression of the multi-chain protein in muscle, in accordance with the present invention, preserves the interaction between the chains and maintains the specificity of the ligand binding site or substrate binding/cleavage site.

The distinction between ligand binding and substrate binding is not absolute. For example, multi-chain proteins that are both ligand binding and substrate binding are known. Illustrative of these is an abzyme. Such multi-chain proteins also can be expressed by muscle and enter the circulation, pursuant to the inventive methodology.

In this description, the terms “polypeptide,” “peptide,” and “protein” are used interchangeably to refer to a polymer of amino acid residues. A ligand or substrate can be any type of organic or inorganic molecule, including but not limited to a protein, a glycoprotein, a proteoglycan, a lipoprotein, a nucleic acid, lipid and combinations thereof. In this regard, the term “nucleic acid” refers to a deoxyribonucleotide or ribonucleotide polymer in either single- or double-stranded form, and also encompasses known analogs of natural nucleotides that can function in a similar manner as naturally occurring nucleotides.

An “antibody” in this context is a protein that is made up of one or more polypeptides, substantially encoded by immunoglobulin genes or fragments of such genes. The recognized immunoglobulin genes include the kappa, lambda, alpha, gamma, delta, epsilon and mu constant region genes, as well as a myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each a tetramer is composed of two identical pairs of polypeptide chains, one pair being a “light” chain (about 25 kD) and one being a “heavy” chain (about 50-70 kD). The N-terminus of each chain defines a variable region of about 100 to 110 or more amino acids primarily responsible for antigen recognition. The terms “variable region of the light chain” (V_{L}) and “variable region of the heavy chain” (V_{H}) refer to these regions of the light and heavy chains, respectively. The antigen-recognition site or ligand/substrate-binding site of an immunoglobulin molecule is formed by three highly divergent stretches within the V regions of the heavy and light chains known as the “hypervariable regions” or “complementarity determining regions (CDRs),” which are interposed between more conserved flanking/connecting stretches known as “framework regions.” In an antibody molecule, the three hypervariable regions of a light chain and the three hypervariable regions of a heavy chain are disposed relative to each other in three dimensional space to form an antigen binding surface. This surface mediates recognition and binding of the target antigen or ligand/substrate. The sequences of many immunoglobulin heavy and light chain hypervariable regions are disclosed, for example, by Kabat et al. SEQUENCES OF PROTEINS OF IMMUNOLOGICAL INTEREST, 4th ed. U.S. Dept. Health and Human Services, Public Health Services, Bethesda, Md. (1987). An “epitope” is that portion of an antigen that interacts with the antibody binding site.

Antibodies exist as intact immunoglobulins or as a number of well-characterized fragments, such as those produced by digestion with various peptidases and those that can be made by recombinant DNA technology. Antibody fragments include Fab′ monomer, Fab′2 dimer, Fv fragment, single chain Fv (“scFv”) fragment, and the like. See e.g., Huston et al., Proc. Nat'l Acad. Sci. USA, 85, 5879 (1988). Antibody fragments also can include unique antibody forms having a truncated, or deleted segment of the light and/or heavy chain constant region. Mutant antibodies may be produced by deletion, truncation, or insertion in the constant or variable regions.

Multi-chain proteins that can be expressed by the method may be modified from those known to occur naturally (e.g., deletions, additions, mutations) or may involve formation from polypeptides that are not known to associate in nature. Multi-chain proteins may comprise at least one polypeptide that is a member of the immunoglobulin superfamily of proteins. The immunoglobulin gene superfamily contains several major classes of molecules. For example, See Williams and Barclay, IMMUNOGLOBULIN GENES (page 361), Academic Press, New York (1989).

The multi-chain proteins expressed in accordance with the methods of the invention that form a substrate binding domain will generally have an association constant for the substrate that is greater than 10³ M⁻¹, more preferably greater than 10⁶ M⁻¹, and even more preferably greater than 10⁷ M⁻¹. The multi-chain proteins expressed in accordance with the methods of the invention that form a ligand binding domain will generally have an association constant for its preselected ligand that is greater than 10⁶ M⁻¹, more preferably greater than 10⁷ M⁻¹, or 10⁸ M⁻¹ and even more preferably greater than 10⁹ M⁻¹.

Nucleic acid encoding each polypeptide of the multi-chain protein can be obtained by methods well known in the art including cloning from cDNA libraries, genomic libraries, and the like. In addition, the sequences of many genes of interest are available in public databases which allows one to synthesize the gene with the aid of DNA amplification techniques such as polymerase chain reaction (PCR). Sequences from public databases also can provide useful information for preparing PCR primers to amplify a particular polypeptide encoding DNA sequence from a suitable cDNA or genomic DNA source. cDNA or genomic DNA may be isolated from cells or tissues of an animal or from cell lines such as from public repositories such as the American Type Culture Collection (Manassas, Va. USA 20108).

Expression of the individual polypeptides of a multi-chain protein can be performed, in accordance with the present invention, by the use of individual expression vectors or a single expression vector. If a single vector is employed for each chain, respectively, the vectors can be mixed before injection and electroporation, in order to allow individual muscle cells to take up and express each of the vectors.

The term “expression-vector” refers to a plasmid, virus, or other vehicle that can be manipulated by insertion or incorporation of a polypeptide-encoding nucleic acid and that is capable of directing the expression of the polypeptide when the vector is in an appropriate environment. A suitable expression vector typically includes a promoter, an origin of replication, a poly-A recognition sequence, and a ribosome recognition site or internal ribosome entry site, and may include other regulatory elements such as an enhancer (tissue specific).

The promoter, which facilitates the efficient transcription of the inserted encoding nucleic acid sequence in muscle, can be constitutive or, if desired, inducible, tissue specific or developmental stage specific. The promoter may be one that normally functions in muscle such as the skeletal actin gene promoter (Muscat et al., Mol. Cell. Biol. 7, 4089 (1987)), the muscle creatine kinase promoter (Sternberg et al., Mol. Cell. Biol. 8, 2896 (1988)), the myosin light chain enhancer/promoter (Donoghue et al., Proc. Natl. Acad. Sci., USA 88, 5847 (1991)), and the like. A promoter not normally associated with expression in muscle may be used, provided that it functions to direct transcription in muscle cells. An example of such as promoter is a viral promoter such as the human CMV promoter, used to express light and heavy chains of an antibody as described in the Examples.

A preferred expression vector comprises an expression cassette that has multiple endonuclease restriction sites allowing ready cloning of DNA encoding different polypeptides into the cassette in a manner that places the encoding DNA in operative linkage with the promoter or other transcriptional regulatory elements of the vector. A preferred expression vector also may have an origin of replication for a procaryotic cell and at least one selective marker to aid in cloning in such cell. One skilled in the art would know how to optimize expression by selecting a vector properly configured with the appropriate combination of promoter, enhancer and other transcriptional or translational regulatory element for the polypeptide(s) to be expressed in muscle.

The inventive methodology allows for multi-chain protein expression from skeletal muscle, smooth muscle, and cardiac muscle, respectively. Expression following injection of skeletal muscle is preferred because of the abundance and ready access of this muscle source. For skeletal muscle, the expression vector may be injected through the skin and into the skeletal muscle via traditional means such as with a syringe and needle, or by a needle-free or needle-less injection device. Such latter devices are well known and, generally, involve pressure-assisted delivery through a tiny orifice held against the skin. For gas-powered, disposable, needle-less hypodermic jet injectors, see U.S. Pat. Nos. 4,596,556 to Morrow et al.; 4,913,699 to Parsons; and 5,730,723 to Castellano et al. Needle-free, gas powered injectors also are available commercially; for instance, see the BIOJECT® device of Bioject Medical Technologies, Inc. (Portland, Oreg.). Another needle-free device is a biolistic delivery device that uses pressurized gas to deliver small particles (e.g., gold particles) to targeted regions of the skin, as a function of the gas pressure. An example of a biolistic delivery device is the PDS-1000 “gene gun” of Dupont (Wilmington, Del.).

Vector can be administered in 0.9% sodium chloride, however, there are a variety of solvents and excipients that may be added without impacting the expression level. For example, it is well known in the art that sucrose is capable of increasing DNA uptake in skeletal muscle. Other substances may also be co-transfected with the vector for a variety of beneficial reasons. For example, P188 (Lee et al., Proc. Nat'l Acad. Sci. USA 89, 4524 (1992)), which is known to seal electropermeabilized membranes, may beneficially affect transfection efficiencies by increasing the survival rate of transfected muscle fibers.

“Electroporation” as used herein means the application of at least one electric pulse to a cell so as to allow transient permeability of a large molecule through the cell membrane. The methods of the invention use electroporation to enhance the level of expression of the multi-chain protein in the circulation of the individual following injection of the vector into muscle. A suitable device and procedure for achieving efficient electroporation of vector following injection of skeletal muscle is provided in U.S. Pat. No. 6,110,161 to Mathiesen et al. As described there, electroporation can be achieved by placing electrodes on the muscle about 1-4 mm apart at the site where the vector is injected. The exact position or design of the electrodes is not critical so long as current is permitted to pass through the muscle fibers in the area of the injected molecule.

Once the electrodes are in position, the muscle is electroporated by administration of one or more square bipolar pulses having a predetermined amplitude and duration. One skilled in the art can optimize the transfection efficiencies for a particular set of circumstances to achieve the desired level of expression. For example, the voltages can range from approximately 0 to 1500 volts depending on the distance between the electrodes, pulse durations can vary from 5 μs to 500 ms, pulse number can vary from one to 30,000, and the pulse frequency within trains can vary from 0.5 Hz to 10,000 Hz. In general, if the field strength is above about 50 V/cm, the other parameters may be varied depending on the experimental conditions desired. In a preferred embodiment, electroporation is achieved by applying about 10 trains of 1,000 pulses each, with each pulse for 400 μs duration at a potential of 150-170 V/cm and with a current limit at 50 mA. Mathiesen, Gene Therapy 6(4), 508 (1999), and Rizzuto, Proc. Nat'l Acad. Sci. USA 96(11), 6417 (1999). The pulses may be monopolar or bipolar. In general short pulse duration can be combined with higher field strength and vice versa. Effective transfection efficiencies are generally obtained with higher field strengths, the field strength being calculated using the formula:

E=V/(2r 1n (D/r)),

which gives the electric field between wires if D>>r. In the formula, V=voltage=10 V, D=distance between wire centers=0.1-0.4 cm, r=diameter of electrode=0.06 cm. See Hofmann, “Cells in electric fields,” in E. Neumann, A. E. Sowers, & C. A. Jordan (eds.), ELECTROPORATION AND ELECTROFUSION IN CELL BIOLOGY, pages 389-407 (Plenum Publ. Corp. 1989). At 10 volts, the field strength is between 163 V/cm-43 V/cm (from 0.1 to 0.4 cm between electrodes, respectively). Because D is not much greater than r, it may be more appropriate to use the formula for electric fields between large parallel plates:

E=V/D

This gives a similar field strength of between 100 V/cm-25 V/cm (from 0.1 to 0.4 cm between electrodes, respectively). The field strength and other parameters are affected by the tissue being transfected, and optimal conditions therefore may vary. For the parameters identified here, in relation to the invention, optimization is a matter of straightforward empirical testing.

In general terms, transfection of vector can be achieved with at least one or more electrical pulses comprising an electrical current having a field strength in the range of from about 25 V/cm up to 200 V/cm. The range also can be 25 V/cm up to 300 V/cm. Transfection also can be achieved by applying a single square bipolar pulse with a duration of between about 50 μs to 5,000 μs, or by delivering multiple square bipolar pulses (2 to about 30,000). In the latter case, the sum of the pulse durations of the bipolar pulses is preferably between about 10 ms to about 12,000 ms. Bipolar pulses can be delivered in the form of at least two trains. The frequency of the electrical stimulation is preferably between about 0.5 Hz and 1000 Hz.

Heart muscle can be transfected by inserting electrodes into the myocardium in the area of injected DNA or by placing the electrodes on or in the outside surface of the heart circumventing the area of the DNA injected myocardium. DNA can be injected from the outside or from the inside of the heart by means of electrodes inserted through the veins or arteries. In this case, the electrode can be hollow, providing a bore through which the vector travels to reach the muscle. Similarly, DNA can be injected into tissue containing smooth muscles and electrical fields can be applied from the outside or inside.

For cardiac or smooth muscle, the electrical field strength should be sufficiently large to permeabilize the cells but less than that which irreversibly damages the tissue. In preferred embodiments, pulsing can be timed to contraction of the heart. For example, voltage can be applied when the heart is contracted during diastole. For this purpose, an electroporator can be used that allows electroporation pulsing to be triggered with the heart rhythm (e.g., an electrocardiogram signal). In general, electrical field strengths in the range of 20-800 V/cm can be applied to cardiac or smooth muscle, while the other pulse parameters may be the same as for skeletal muscle.

In addition to electrical parameters, the desired level of expression of a multi-chain protein can be affected by a variety of approaches, including, for example, by increasing the numbers of muscle sites injected, increasing the amount of plasmid used per injection, reducing immunogenicity by removing xenogenic or allogeneic antigenic determinants from the protein, or by using Ig constructs, vectors or promoters further optimized for expression. The choice of longer half life immunoglobulin in a particular setting (e.g., human IgG in a human) can also be used to increase the concentration of muscle expressed antibody in an individual. Skeletal muscle from anywhere in the body can be used for this purpose, as can cardiac or smooth muscle. Furthermore, any individual with muscle can be used in the method, including animals, such as mammals, (e.g., humans, goats, sheep, cattle, and the like).

As shown herein, the level and persistence of heteromultimer in the serum is affected by the protein's immunogenicity. It was discovered herein that fully murine antibody or partially chimeric murine antibody (light chain constant region only) expressed from muscle in mice persisted longer in serum (months as opposed to weeks) than a fully chimeric form of the antibody (both heavy and light chain human constant regions). Small amounts of foreignness like mouse IgG2b^{a} allotype and human Cκ were apparently accepted without seriously compromising long term serum expression.

However, one may take advantage of immunogenicity to raise antibodies in the individual to the expressed multi-chain protein. Accordingly, a method is provided for obtaining antibodies to a protein that comprises at least two different polypeptide chains and wherein the protein comprises one or more antigenic determinants foreign to the individual. The method comprises injecting into muscle of an individual at least one expression vector that encodes the polypeptide chains. In accordance with this method, uptake of the vector into muscle cells results in secretion of the protein. The resulting antibodies can be obtained from the individual.

The present invention makes possible various approaches for generating an immune response to an antigen. The antigen to which an immune response is generated may have a single foreign epitope or may have multiple foreign epitopes. Such response may be used to protect the individual from infectious microbial agents such as bacteria, fungi, protozoa virus, and the like, without having to expose the individual to the infectious agent. The methods of immunization also can be used to protect the individual from cancer or at least delay the onset of disease or delay death. A variety of well known tumor associated antigens exist which can be used to elicit an immune response in humans as disclosed herein. Such antigens include carcinoembryonic antigen (CEA), idiotypic (Id) determinants on monoclonal immunoglobulins, and the like.

By one approach, the antigen can be expressed physically associated with at least one of the polypeptide chains of the multi-chain protein. This can be conveniently achieved by placing the DNA encoding the antigen at one or both the ends of the DNA encoding one or both chains. In the case where the multi-chain protein is an antibody, the antigen can be expressed as a fusion to the either or both the N and C-terminus of the light and/or heavy chain of the antibody. The DNA encoding the antigen, however, may also be placed within a sequence encoding an antibody heavy or light chain.

Accordingly, a method of immunizing an individual is provided, the method comprising injecting at least one expression vector into the muscle of the individual, the vector comprising nucleic acid encoding an antibody fusion protein, the fusion protein comprising an antibody specific for a cell surface marker of an antigen presenting cell of the individual, the antibody fused to a polypeptide antigen to which immunization is desired. In accordance with the method, uptake of the vector into muscle cells results in secretion of the antibody fusion protein, the secreted fusion protein functioning to target the antigen to the surface of antigen presenting cells of the individual. In a preferred embodiment, one or more electrical pulses is applied to the muscle at the site of injection to enhance expression of the protein. Although not wishing to be bound by any theory, the inventors believe that antibody produced and released by muscle cells can circulate and bind to the cell surface of antigen presenting cells, thereby concentrating antigen physically associated with such antibody on the cells critical to initiation of an immune response. Furthermore, the binding to, for example, an MHC class I molecule could induce anergy or tolerance to the antigen. In preferred embodiments, the cell surface marker is an MHC class II molecule, B7 molecule, IgD, Fc-receptor, CD40, or Toll receptor.

In another approach, the antigen is associated with the light chain or heavy chain of a bispecific antibody. A “bispecific antibody” is a four chain antibody with two binding site, where one site is specific for one antigen and the other site is specific for another antigen. In some cases, the bispecific antibody may have two different light chains and two different heavy chains or it may have a common light chain or a common heavy chain, but not both. In this immunization approach, the bispecific antibody has a binding site specific for a cell surface marker of an antigen presenting cell of the individual, and a binding site specific for the antigen. One or more vectors may be used to encode the bispecific antibody heavy and light chains and an immune response is elicited by injecting the expression vector(s) into the muscle of the individual and applying at least one electrical pulse to the injection site.

Accordingly, a method is provided for immunizing an individual, comprising injecting at least one expression vector into the muscle of the individual, the vector encoding the light chains and the heavy chains of a bispecific antibody, the bispecific antibody having a first binding site specific for a cell surface marker of an antigen presenting cell of the individual and a second binding site specific for an antigen to which immunization is desired. In accordance with this method, uptake of the vector into muscle cells results in secretion of the bispecific antibody in the circulation of the individual. In furtherance of the method, antigen is administered to the individual so that the antigen is targeted to antigen presenting cells by the bispecific antibody. In a preferred embodiment, one or more electrical pulses is applied to the muscle at the site of injection to enhance expression of the protein. In preferred embodiments, the cell surface marker is an MHC class II molecule, B7 molecule, IgD, Fc-receptor, CD40, or Toll receptor.

In another approach, the antibody can be fused to a signaling protein that can exert an effect intracellularly. Such signalling proteins are well known in the art and include, for example, regulatory proteins for gene expression, proteins involved in intracellular signaling pathways (i.e. apoptotic signal), proteins that increases intravesicular pH in the endosomes where the mAb-fusion protein is transported, and the like.

Antigen can be administered to the individual by administration intravenous (iv), intramuscular (im), subcutaneous (subQ), intraperitoneal (ip), orally or by any other known route. Administration can be effected by any means known in the art including traditional means such as using a syringe and needle, or by a needle-free or needle-less injection device. Such latter devices are well known and, generally, involve pressure-assisted delivery through a tiny orifice. Gas-powered, disposable, needle-less hypodermic jet injectors or needle-free, gas powered injectors can be used. Antigen also can be administered using a biolistic delivery device that uses pressurized gas to deliver small particles (e.g., gold particles) to targeted regions of the muscle, as a function of the gas pressure. An example of a biolistic delivery device is the PDS-1000 “gene gun” of Dupont (Wilmington, Del.).

Again, without ascribing to a particular theory, the inventors believe that bispecific antibody expressed by muscle cells reaches the circulation and binds to the surface of antigen presenting cells by virtue of one binding site of the bispecific antibody. Antigen administered to the individual can be concentrated on the surface of such antigen presenting cells, thereby enhancing the immune response to the antigen.

The antigen may be administered as a solution formulated with a buffer or other suitable fluid. The antigen also may be administered with an adjuvant either by mixing the antigen with the adjuvant or by conjugating or otherwise linking the antigen to the adjuvant. A variety of adjuvants are known including Freund's (complete and incomplete), alum, muramyl dipeptide, BCG, LPS, Ribi Adjuvant System®, TiterMax®, and the like. One skilled in the art would know which type of adjuvant is appropriate to use in a given circumstance.

In another embodiment, instead of administering the antigen, an expression vector is used to encode the antigen, which is co-expressed with the bispecific antibody by the same approach of muscle injection and electroporation. The antigen may be encoded by an expression vector that is separate from the expression vector(s) used to encode the antibody chains, or the antigen may be encoded by a vector that also encodes at least one of the antibody chains. The antigen and the antibody may be transfected in the same or different muscles in the same animal.

In the various above embodiments, the antibody can include any of the binding specificities of antibodies which have been approved for clinical use (See, e.g., Table 1).

TABLE 1
List of Approved Therapeutic Monoclonal Antibodies

| Year | | Target | | | Type of |
|---|---|---|---|---|---|
| Approved | Product Name | Antigen | Company | Indication | Antibody |
| 1986 | Orthoclone | T-lympho- | Ortho Biotech, | Organ | Murine Mab |
| | OKT3, | cyte surface | Raritan, NJ | transplant | |
| | muromonab- | antigen CD3 | | rejection | |
| | CD3 | | | | |
| 1994 | ReoPro, | Platelet | Centocor, | Coronary | Fab derived |
| | abciximab | surface | Malvern, PA | intervention | from chimeric |
| | | receptor | | and | Mab |
| | | gpllb/IIIa | | angioplasty | |
| 1995 | Panorex, | 17-1a, | Centocor, | Colorectal | Murine Mab |
| (Germany | edrecolomab | EpCAM | Malvern, PA | cancer | |
| only) | | | | | |
| 1997 | Rituxan; | B-cell | IDEC Pharma, | Non- | Chimeric MAb |
| | rituximab | surface | San Diego, CA | Hodgkin's | |
| | | antigen | | lymphoma | |
| | | CD20 | | | |
| | ReoPro, | Platelet | Centocor, | Refractory | Fab from |
| | Abciximab | receptor | Malvern, PA | unstable | chimeric Mab |
| | | gpIIb/IIIa | | angina | |
| | Zenapax, | IL-2 | Protein Design | Kidney | Humanized |
| | Daclizumab | receptor [symbol] - | Labs, | transplant | Mab |
| | | chain | Fremont, CA | rejection | |
| | | (CD25) | | | |
| 1998 | Herceptin, | Human | Genentech, | Metastatic | Humanized |
| | trastuzumab | epidermal | S. San | breast cancer | Mab |
| | | growth | Francisco, | | |
| | | factor-like | CA | | |
| | | receptor-2 | | | |
| | | (HER-2) | | | |
| | Remicade, | TNF- [symbol] | Centocor, | Crohn's | Chimeric MAb |
| | infliximab | | Malvern, PA | disease | |
| | Simulect, | IL-2 | Novartis | Kidney | Chimeric MAb |
| | basiliximab | receptor [symbol] - | Pharma, East | transplant | |
| | | chain | Hanover, NJ | rejection | |
| | Synagis, | Respiratory | MedImmune, | Respiratory | Humanized |
| | Palivizumab | syncytial | Gaithersburg, | syncytial virus | Mab |
| | | virus | MD | disease | |
| | | surface | | | |
| | | antigen “F” | | | |
| | | protein | | | |
| 1999 | Remicade | TNF- [symbol] | Centocor, | Rheumatoid | Chimeric |
| | Infliximab | | Malvern, PA | arthritis | Mab |
| 2000 | Mylotarg, | CD33 | Celltech | Chemothera- | Humanized |
| | Gemtuzumab | | Chiroscience, | peutic MAb for | MAb |
| | Ozogamicin | | Slough, UK | CD33 positive | conjugated to |
| | | | and | acute | calichemicin |
| | | | Wyeth-Ayerst | myeloid | |
| | | | (American Home | leukemia in | |
| | | | Products), | relapsed and | |
| | | | Philadelphia, PA | older patients | |
| 2001 | Campath ®, | B- | Millennium, | B-cell chronic | Humanized |
| | alemtuzu-mab | lymphocytes | Cambridge, MA | Lymphocytic | Mab |
| | (humanized | | and | leukemia (B- | |
| | Monoclonal | | Ilex Oncology | CLL) | |
| | antibody) | | San Antonio, TX | | |

The invention methods also can be used to test a biological property of a recombinant antibody without having to prepare transformed or transduced cell lines that express the antibody. Accordingly, muscle of an individual is injected with at least one expression vector that encodes the heavy and light chain of the antibody, such that uptake of the vector into muscle cells results in secretion of the antibody. The biological property may be evaluated within the individual in situ without requiring removal of the expressed antibody or expressed antibody can be obtained from the individual and then tested for a biological property elsewhere. For example a mAb directed to a particular tumor associated antigen could tested for the biological property of tumor therapy without removing the expressed antibody from the individual provided that the individual carries the appropriate tumor. In a preferred embodiment, one or more electrical pulses is applied to the muscle at the site of injection to enhance expression of the protein. In some embodiments, the DNA encoding the antibody heavy and light chains may be mutated by means well known in the art including, for example, addition, deletion, truncation and point mutation. The effect of the mutations on antigen binding specificity can then be evaluated by expressing the protein in accordance with the method.

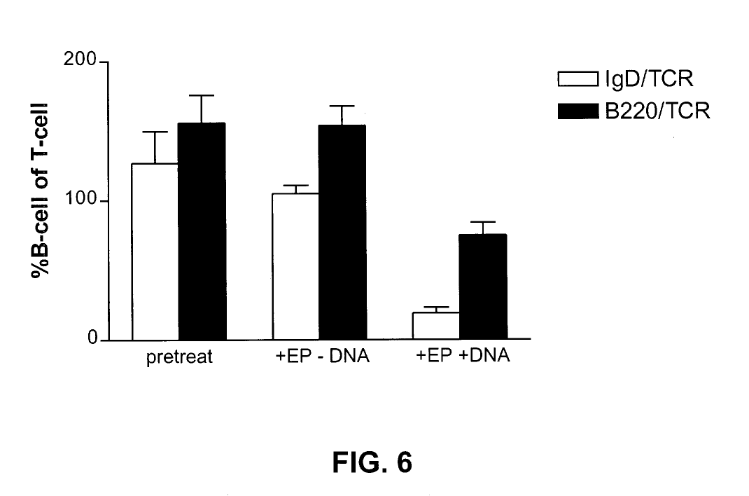

In the case of immunoglobulins, biological properties to be tested include, for example, antigen specificity, complement activation, Fc-receptor mediated phagocytosis, induction of signaling cascades, and the like. By expressing genes directly from the muscle of an individual, recombinant mAbs can be screened more efficiently and with less time. The ability of recombinant expressed antibodies to exhibit biological activity in vivo can be evaluated using the method of the invention. Example 4 (FIG. 6) shows that expressing an antibody specific for a B cell surface marker (IgD) in accordance with the method is effective in depleting this population of cells in vivo. Other types of biological activity manifest in vivo also may be similarly evaluated.

Those skilled in the art will recognize that various modifications can be made to the present invention without departing from the spirit and scope thereof. The invention will now be described in greater detail by reference to the following non-limiting examples.

### EXAMPLES

### Example 1

Serum Expression of a Chimeric Human/Mouse Antibody Specific for I-E^{d} or IgD^{a} following Electroporation of Expression Vectors in Muscle

This example demonstrates that expression vectors coding for a two chain structure, in this case, a heavy and a light chain of an antibody molecule, can be injected into skeletal muscle, resulting in production of an intact complete antibody and release of the antibody into the circulation of the individual. An experiment was designed to evaluate expression of full sized antibody following electroporation of muscle injected either with a mixture of vectors, each coding for the heavy chain or the light chain of an antibody, or one vector encoding both types of chains. In this regard, three expression vectors were prepared; A heavy chain vector construct was made containing DNA encoding a chimeric heavy IgG3 (mouse V gene and human C gene); a light chain vector construct was made containing DNA encoding a chimeric human kappa light chain (mouse V gene and human C gene); and a combination vector (“combi” vector) was made that contained DNA encoding both the chimeric heavy and the chimeric light chain.

A. Antibody V_{H} and V_{L} Regions

The V genes of the light and heavy chains were obtained from the 14-4-4S (ATCC designation “HB32”) mouse hybridoma, which produces a mouse IgG2a kappa antibody specific for the alpha chain (determinant Ia.7) of the I-E MHC class II molecule of mice. Ozato et al., J. Immunol. 124:533 (1980). 14-4-4S, therefore, is a mouse IgG2a antibody specific for I-E^{d}.

Nucleic acids encoding the V domains of the light and heavy chain of 14-4-4S were cloned, essentially as described previously by Norderhaug et al., J. Immunol. Methods 204, 77 (1997), with respect to the TP-3 antibody. Briefly, cDNA was prepared from the 14-4-4S hybridoma and the V_{L} and V_{H} genes were amplified using a set of degenerate upstream primers that anneal in the various immunoglobulin leader sequences in combination with downstream primers annealing to CH1 for the heavy chain or C kappa for the light chain. The PCR products were then sequenced, and specific PCR primers annealing to the exact ends of the cloned V regions were designed. These primers were designed to include restriction enzyme sites (underlined and bolded), the upstream V_{L} primer has a BsmI site and the downstream primer has a BsiWI site, while the upstream V_{H} primer has an MfeI site and the downstream V_{H} primer has a BsiWI site. The primer sequences are as follows:

5′ V_{L} (upstream):

(SEQ ID NO:1)

5′-GGTGTGCATTCCGACATTGTTCTGACACAGTCTCC-3′
3′ V_{L} (downstream):

(SEQ ID NO:2)

5′-ACGTACGTTCTACTCACGCTTGATTTCCAGCTTGGTGCC-3′
5′ V_{H} (upstream)

(SEQ ID NO:3)

5′-CAGGTCCAATTGCAGCAGTCTGG-3′
3′ V_{H} (downstream)

(SEQ ID NO:4)

5′-GACGTACGACTCACCTGAGGAGACCGTGACTGAGGTT-3′

The nucleotide sequences encoding 14-4-4S V_{L} and V_{H} regions are available in the EMBL GenBank public databases under accession numbers AF292646 and AF292391, respectively.

The V_{L} and V_{H} regions from the Ig(5a)7.2 hybridoma, which produces a mouse monoclonal antibody with specificity for the allotype of murine IgD (IgD^{a}) were obtained essentially as described by Lunde et al., Nature Biotechnology 17, 670 (1999).

B. Antibody Expression Vectors

Antibody chain expression shuttle vectors pLNOH2 and pLNO_{κ} were prepared as described by Norderhaug et al., J. Immunol. Methods 204, 77 (1997).

V_{L} of 14-4-4S prepared as described above was cut with BsmI and BsiWI and cloned in pLNO_{κ} similarly digested to yield a chimeric human/mouse kappa light chain with a V_{L} region from the 14-4-4S antibody and the human kappa constant region from the vector (14-4-4S V_{L}/human C_{κ}). V_{H} of 14-4-4S prepared as described above was cut with MfeI and BsiWI and cloned in pLNOH2 similarly digested to yield a human chimeric human/mouse gamma 3 heavy chain with the V_{H} region from the 14-4-4S antibody and the human gamma 3 constant region from the vector (14-4-4S V_{H}/human gamma 3). These 14-4-4S chimeric antibody heavy and light chain shuttle vectors are described in Lunde et al. J. Immunol. 168, 2154-2162 (2002).

The gamma 3 heavy chain sequence used in the chimeric 14-4-4S was mutated, as described by Lunde et al., Molecular Immunology 34, 1167 (1997), so as to encode an 11-amino acid, tumor-specific T cell epitope from the mouse myeloma protein of the MOPC 315 tumor. See Bogen and Weiss, Int. Rev. Immunol. 10, 337 (1993); Bogen et al., Eur. J. Immunol. 16, 1373 (1986); Bogen et al., loc. cit. 16, 1379 (1986). This mutation does not influence secretion and folding of the antibody. Lunde et al. supra, 2002.

The combi vector was prepared as described in Norderhaug et al., J. Immunol. Methods 204, 77 (1997). Briefly, the CMV promoter, V_{L} and Cκ were isolated from pLNOK/14-4-4S V_{L} as a 2.6 kb BglII-BamHI fragment and subcloned into an alkaline phosphatase treated BamHI restriction site of pLNOH2.

Vectors encoding a human/mouse chimeric anti-mouse IgD^{a} antibody was prepared in the same way as the anti I-E^{d} antibody vectors except that the variable regions of the heavy and light chains were cloned from the Ig(5a)7.2 hybridoma, which produces a mouse monoclonal antibody with specificity for the allotype of murine IgD (IgD^{a}). See Lunde et al., Nature Biotechnology 17, 670 (1999). Briefly, V_{L} of Ig(5a)7.2 (IgD^{a} antibody) was cloned in pLNO_{κ} to yield a chimeric human/mouse kappa light chain with a V_{L} region from the Ig(5a)7.2 antibody and the human kappa constant region from the vector Ig(5a)7.2 (V_{L}/human C_{κ}). V_{H} of Ig(5a)7.2 was cloned into pLNOH2 to yield a chimeric human/mouse gamma 3 heavy chain with the V_{H} region from the Ig(5a)7.2 antibody and the human gamma 3 constant region from the vector Ig(5a)7.2 (V_{H}/human gamma 3).

C. Injection and Electroporation

Vector DNA (100 μg) was injected i.m. into the quadriceps of various mice including Balb/c mice (positive for MHC class II I-E^{d}), C57BL mice negative for MHC class II I E^{d}), B10-D2 mice, BALB.B mice and C.B.-17 mice. Vector DNA diluted in 0.9% NaCl was injected into both quadriceps (50 μg/50 μl/quadriceps). One group of mice were injected with the combi vector while another group of mice were injected with a mixture of the separate heavy and light chain vectors. Electroporation was performed following injection, by applying electrodes to the muscle at the site of the injection and subjecting the site to an electrical potential comprising 10 trains of 1000 pulses each, with a pulse length at two times 200 μSec (positive 200 μSec and negative 200 μSec) with 600 μs interval between each pulse with a current limit of 50 mA (about 150-174 V/cm). Each train is separated by a one second interval. Conductive gel was used at the skin. In larger animals, the electrodes may be inserted into the muscle.

D. Assay

mAb levels were determined by ELISA. See, e.g., Lauritzsen, et al., Scand. J. Immunol. 33, 647-656 (1991). Briefly, plastic microtiter plates (Costar Polystyrene High binding) were coated at least overnight at 4° C. by addition of 50 μl monoclonal anti-human IgG3 (Sigma, 1-9763) (for detection of chimeric human IgG3 mAb, both anti-I-E^{d} and IgD) at 1:5000 dilution in PBS with azide and the wells were blocked from further nonspecific binding by treatment with PBS containing 0.5% BSA (at least 10 minutes incubation at RT). The plates were then washed by rinsing the wells 4 times in washing buffer (0.1% Tween 20 in PBS).

Blood samples obtained from the mice at various days (e.g., 0, 7, 14, 21 and 28) were allowed to clot. Serum was separated and diluted 1:5 in PBS containing 0.2% BSA and 0.2% Tween 20 (dilution buffer). Diluted serum samples were added to the blocked microtiter assay plates and incubated at 37° C. for 1 hr. Human IgG3 (Sigma, I-4389) was used as standard. The wells were washed four times in washing buffer, biotinylated anti-human IgG3 (Sigma, B-3523; 1:2000 in dilution buffer) or biotinylated anti human kappa antibody (Sigma, B-1393) added and the plate was incubated overnight at 4° C. The wells were washed four times in washing buffer and streptavidin-alkaline phosphatase conjugate (Amersham Life Science; diluted 1:3000 in dilution buffer) added and the plate incubated for at 37° C. for 1 hr. After final washing, phosphatase substrate (Sigma, p-Nitrophenyl Phosphate, Disodium, 5 mg/tablet, use 1 mg/ml) was added and the plate incubated for 10-30 min at room temperature. The optical density at 405 nm was determined.

E. Results

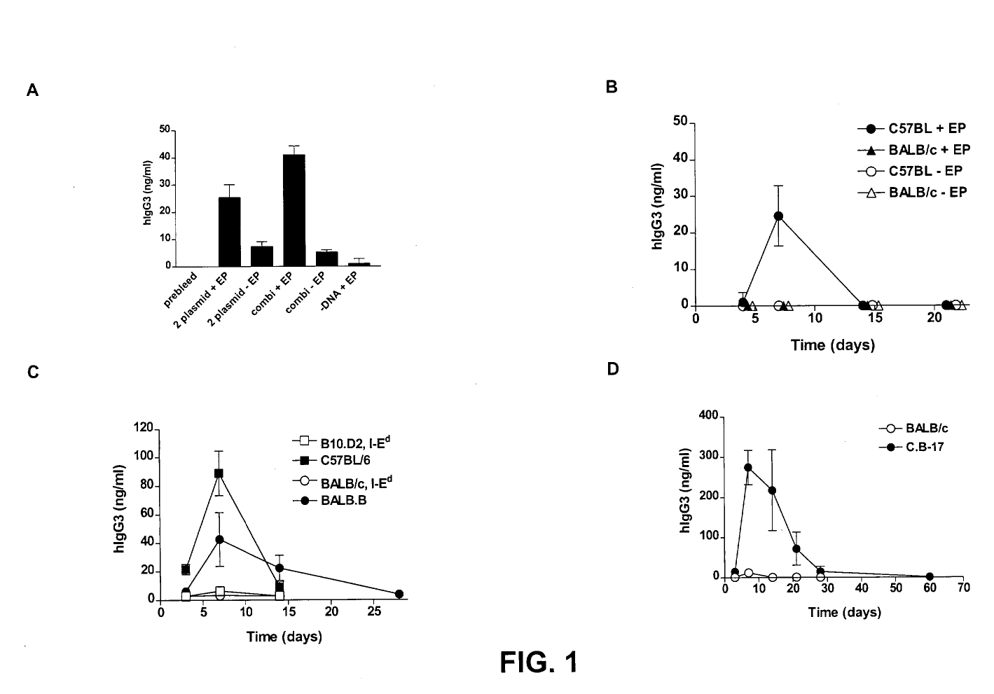

The amount of chimeric antibody as shown by detecting the antibody heavy chain in the serum of C57BL/6 mice is shown in FIG. 1. Chimeric antibody was first detected in serum at around day 3-6 after electroporation. Co-injection of separate plasmids for chimeric anti-I-E^{d} H- and L-chain genes as well as injection of the single “combi” plasmid containing both the heavy and light chain, induced only very low amounts of serum mAb hardly detectable at all (FIG. 1A). However, when injection of plasmid DNA was followed by in vivo electroporation consisting of low voltage, high frequency electrical pulses applied to the skin over the injection site, levels of serum mAb were considerably increased (FIG. 1A). Thus, electroporation enhanced production of mAbs from Ig-genes injected as naked DNA plasmids. Moreover, Ig H-and L-chain genes did not need to be on the same plasmid for in vivo expression to ensue. The antibody consisted of both heavy and light chain shown with ELISA. Detection of heavy and light chain in the same ELISA gave similar results as that for the heavy chain shown in FIG. 1A.

Evidence that the expressed chimeric antibody retained its antigen binding specificity was obtained by transfecting mice with different genetic backgrounds to determine if tissue expression of I-E^{d} (the antigen detected by the anti-I-E^{d} mAb) affected the level of expressed chimeric antibody detectable in the circulation. For this purpose, the combi vector encoding both the gamma 3 chimeric heavy chain derived from antibody 14-4-4S and a human/mouse chimeric light chain derived from antibody 14-4-4S was electroporated into skeletal muscle of I-E^{d} positive mice (Balb/c) and I-E^{d} negative (C57BL/6) and the level of chimeric antibody in the circulation determined. FIG. 1B shows that chimeric I-E^{d} antibody was detectable in the C57BL/6 mice not expressing I-E^{d} but was not detectable in BALB/c mice that express this antigen. The absence of antibody in mice expressing the antigen suggests that the produced antibody is specific for the antigen.

To prove that the controlling factor in these experiments was I-E^{d} expression and not some other genetic feature of the mouse strain, muscle injection/electroporation with the 14-4-4S H and L vectors was tested on MHC-congenic BALB.B mice, which are identical to BALB/c except that they have the MHC H-2b haplotype and thus lack I-E^{d}, and congenic B10.D2 mice, which are close to identical to C57Bl/6 except that B10.D2 have the H-2d haplotype and express I-E^{d}. As seen in FIG. 1C, mAb was detectable in serum of BALB.B (I-E^{d} negative) but not from the serum of BALB/c mice that express I-E^{d}. Similarly, FIG. 1C shows that mAb was detectable in serum of C57BL/6 mice that lack I-E^{d} but not from the serum of B10.D2 mice that express I-E^{d}. These results showing increased serum 14-4-4S antibody in mice that do not express I-E^{d} demonstrate that the serum expressed 14-4-4S chimeric antibody mAb is specific for its antigen (I-E^{d}).

Similar results were obtained following expression of the IgD^{a} antibody where the variable H- and L chains are derived from the Ig(5a)7.2 hybridoma (FIG. 1D). In this case two separate plasmids encoding the L- and H-chains were co-injected into skeletal muscle and electroporation applied. Human IgG3 ELISA showed chimeric IgD^{a} antibody in serum of C.B-17 mice, which lack IgD^{a} and express IgD^{b}. By contrast, chimeric IgD^{a} antibody was not detected in serum of BALB/c mice, which express IgD^{a} but are otherwise close to identical to C.B-17. Serum levels of anti-IgDa mAb in C.B-17 mice reached about 300 ηg/ml, which was considerably higher than for the anti-I-E^{d} mAb. These results showing increased serum Ig(5a)7.2 antibody in mice that do not express IgD^{a} allotype demonstrate that the serum expressed Ig(5a)7.2 chimeric antibody is specific for its antigen (IgD^{a} allotype).

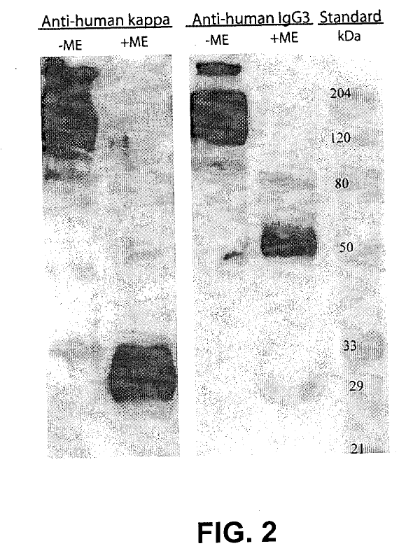

As the binding of mAb to antigen depends on correct association of H and L chains, the results in FIGS. 1B-D support that muscle cells secrete mAb as assembled tetramers with correct specificity and that the mAb reaches distant tissues where it is absorbed by the target antigen expressing cells or tissue. These conclusions are supported by physical analysis of immunoglobulin in the sera of electroporated animals. In brief, Protein G Sepharose beads were incubated with serum from mice co-administered the expression vectors encoding the heavy and light chains of the chimeric anti-IgD^{a} mAb by i.m. muscle injection, followed by electroporation at the injection site. The isolated antibodies were eluted and either treated or not treated with mercaptoethanol in order to separate the heavy and light chain. After gel-electrophoresis and blotting, the blots were developed using either anti-human IgG3 or anti-Cκ. antibodies. The Western blot in FIG. 2 shows that human gamma 3 heavy chain in the sera of mice is associated with a kappa light chain. A similar conclusion was obtained from a sandwich ELISA which identified heavy and light chain markers on the same molecules captured from the serum of treated animals (the ELISA used anti-human γ3 as coat antibody and anti-human Cκ as detection antibody).

The abrupt decline in I-E^{d}-specific serum mAb seen in C57Bl/6 mice between days 7 and 14 as seen in FIGS. 1B and 1C could be caused by an immune response against the xenogeneic parts of the mAb, namely human γ3 and Cκ (table 1). To evaluate this possibility, an ELISA was prepared using plates coated with human-IgG3 (Sigma, I-4389), with detection of anti-Ig antibodies by biotinylated anti-mouse IgG1 or biotinylated anti-mouse IgG2a). Otherwise, the ELISA was the same as before except that the serum was serial diluted.

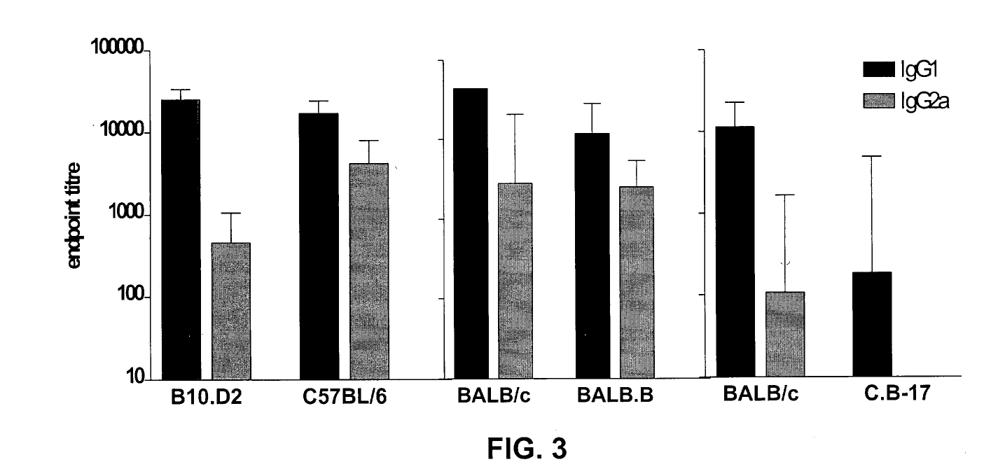

The results showed that the decline of anti-I-E^{d} mAb was paralleled by an increase in mouse anti-human IgG3 antibodies of both IgG1 and IgG2^{a} subclasses, with high serum titers being detected at 28 days after injection (FIG. 3). Induction of mouse anti-human IgG3 mAb was not only detected in I-E^{d} negative strains that had high serum levels of mAb but also positive strains where little or no serum mAb was detected. (FIGS. 3A and B). This result indicates that the chimeric I-E^{d}-specific mAb was produced in sufficient amounts in I-E^{d} positive strains to immunize the mice despite the fact that the chimeric antibody appear to have been quickly absorbed from the serum. Anti-immunoglobulin also was present in the serum of mice at day 28 post transfection with the chimeric anti-IgD^{a} mAb (FIG. 3C). Notably, the C.B-17 mice produced less anti-human IgG3 antibodies especially of IgG2a subclass as compared to the mice that transfected with the I-E^{d} antibody. The differences could be related to the higher serum concentration of anti-IgD^{a} chimeric mAb, use of different V-regions, or an influence of Balb/c background genes.

### EXAMPLE 2

Long Term Serum Expression of Antibodies in vivo is Achieved by Removing Xenogeneic Sequence

Xenogenic sequences were removed from the expressed monoclonal antibodies to determine if reduced immunogenicity would increase the amount or extent of time that recombinant antibody was expressed in the serum. In a first experiment, the heavy chain vector for the chimeric IgD^{a} antibody (where V_{H} is derived from the Ig(5a)7.2 and C_{H} is a human gamma 3 chain) was modified by removing the human gamma 3 constant region and replacing it with the mouse gamma 2b constant region. The resulting vector pLNOH2γ2bV_{H}T (Lunde et al. supra, 1999) with the variable region from Ig(5a)7.2 was co-injected into muscle with the corresponding human/mouse chimeric light chain vector (pLNO_{κ}V_{L}T; Lunde et al. supra, 1999). The expressed antibody is thus partially chimeric with a full mouse heavy chain and a chimeric human/mouse light chain.

Serum analysis was done using by coating NIP_{2.6}BSA to the wells followed by binding of mouse IgD anti-NIP, obtained from cell transfectants. Serum samples are applied and binding determined using anti-mouse IgG2b-biotin.

The results with the mouse C_{H}/human C_{κ} IgD^{a} specific antibody showed that subsequent electroporation was necessary for obtaining reasonably detectable expression, and that the best expression was achieved in mice that do not express the IgD^{a} allotype target antigen (i.e., C.B-17 mice). In the case of the C.B-17 mice, serum mAb were as high as 750 ηg/ml after 1-5 weeks and then declined slowly. Even after 7 months, mice had ^{˜}300 μg/ml in their sera. The fully chimeric version of this antibody showed lower maximum expression and declined more rapidly (compare FIG. 1D). Thus, by eliminating xenogenic parts of the antibody, increased and prolonged the presence in serum was achieved.

A similar experiment was performed by expressing a mouse IgG2b antibody specific for the hapten NIP. The V_{H} gene of the murine NIP specific antibody cloned into vector pSV2gptV_{NP} (Neuberger, EMBO J. 2, 1373 (1983)) was cloned upstream of a human gamma 3 heavy chain to form pLNOH2 (Norderhaug et al. J. Immunol. Methods 204, 77 (1997)). As described in Eidem et al., J. Immunol. Methods 245, 119 (2000), vector pLNOH2-gamma2b was obtained by substituting he human IgG3 encoding sequence in pLNOH2 with a mouse IgG2b constant chain sequence, the latter from the BALB/c-derived myeloma cell line MPC-11. See Lang et al., Nucleic Acids Res. 10, 611 (1982). The DNA encoding a murine lambda light chain for NIP (Celltech Limited) was cloned into an expression vector which was co-electroporated with pLNOH2-γ2b into muscle. The result was production of a full murine NIP specific antibody with constant regions from λ for the light chain γ2b for the heavy chain.

Electroporation into the muscle of Balb/c mice was performed as described in Example 1. Serum was obtained from mice at day 0, 3, 7, 14, and at week 4, 5, and 8 following electroporation. ELISA analysis of serum was performed essentially as described in Example 1 except that plates were coated with NIP₂₆BSA (i.e., 2.6 NIP molecules per BSA molecule), and the detection antibody was anti-IgG2b-biotin (Pharmingen, cat. no: 02032D). Anti-NIP antibodies produced by hybridoma cell lines were purified and used as a standard. The hybridoma cells express the lambda 1 gene and were prepared to express a functional NIP specific antibody by transfection with the same NIP-specific heavy chain construct used for electroporation of mouse muscle. See Eidem, J. Immunol. Methods 245, 119 (2000).

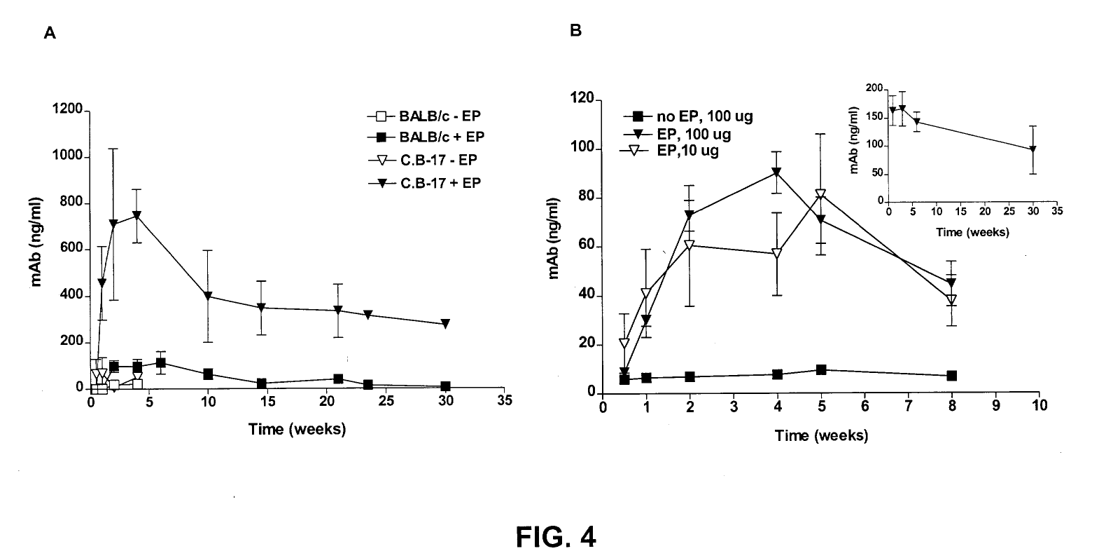

The amount of NIP antibody detected in the serum of injected and electroporated BALB/c mice in two separate experiments (FIG. 4B and insert graph) had significant amounts of anti-NIP mAb in their sera measured by their ability to bind NIP-BSA in an ELISA, with maximal amounts of 60-100 ηg/ml being detected between 2 and 5 weeks. Levels of serum antibody declined slowly but as much as 50 ηg/ml was still detected 30 weeks after DNA injection (FIG. 4B, insert). Electroporation was required for detection of mAb in serum. Injection of 10 and 100 μg plasmid (50 μg and 5 μg, respectively, in each quadrisep) gave similar results but the lower amount showed higher variability (main graph). Mice in the insert graph of FIG. 4B were injected with 50 μg total vector.

### EXAMPLE 3

Complement Mediated Cell Lysis by Serum Expressed Antibody

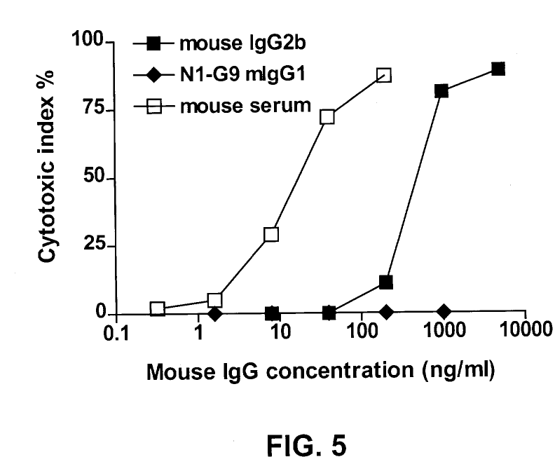

The integrity of the expressed antibody was evaluated for the constant region by determining if the serum expressed antibody bound to its antigen had the ability to activate complement. A complement mediated cells lysis (CML) assay was performed as described. Michaelsen, et al., Scand. J. Immunol. 32, 517-528 (1990); Aase, et al., J. Immunol. Methods 136, 185-191 (1991). Briefly, ⁵¹CR labeled sheep red blood cells (SRBC) were sensitized with NIP by incubating the cells with rabbit anti-SRBC NIP-₁₅-Fab′ fragments. Serial dilutions of the NIP murine antibodies expressed in serum in accordance with the invention were added to the NIP-sensitized ⁵¹CR SRBC. Human serum was used as the complement source. The same NIP antibody expressed from recombinant cells and purified was used as a control. Cytotoxic index (CI) was calculated according to the formula: %CI=[(cpm test−cpm spontaneous)/(cpm max−cpm spontaneous)]×100. The results in FIG. 5 shows that anti-NIP mAb from the serum of vector-injected and electroporated mice was capable of activating complement resulting in red cell lysis. The results show that the Fc region of the NIP mAb produced by muscle is functional in its ability to activate complement.

### EXAMPLE 4

Complement Mediated Cell Lysis by Serum Expressed Antibody

BALB/c mice were injected or not injected with DNA encoding anti-IgD and electroporated. After 7 days, blood was collected in heparin solution to avoid clotting. Lysis buffer (Becton Dickinson) was added to the sample to lyse red blood cells. Cells were washed and resuspended in staining buffer (PBS and 0.5%BSA) containing different antibodies against cell markers. Antibodies used were FITC-IgD, PerCP-B220/CD45R and PE-TCRCbeta, specific for IgD and B200 (on B-cells) and the T-cell receptor (on T-cells), respectively. Following staining, cells were washed, resuspended in fixation buffer (PBS and 2% paraformaldehyde) and analyzed by flow cytometry.

The results are presented as %B-cells of T-cells in the blood of mice treated or not treated with the method. Five mice were used in each group. As can be seen in the figure, IgD and B220 positive cells were depleted in blood when mice had been administered the vector and given electroporation. In this case, the expressed antibody was biologically active in the individual following production by muscle.

What is claimed is:

1. A method of producing a protein in the circulation of an individual, said protein comprising at least two different polypeptide chains, said method comprising injecting into muscle of the individual at least one expression vector that encodes the polypeptide chains, such that uptake of the vector into muscle cells results in production of the polypeptide chains and secretion of the protein.

2. The method of claim 1, wherein said protein is an immunoglobulin.

3. The method of claim 2, wherein said immunoglobulin is an antibody.

4. The method of claim 2, wherein said vector codes for each of a full-length light chain and a full-length heavy chain.

5. The method of claim 1, wherein said individual is a human.

6. The method of claim 2, wherein said immunoglobulin has constant region sequence from human immunoglobulin.

7. The method of claim 2, wherein said immunoglobulin has variable region sequence from human immunoglobulin.

8. The method of claim 1, wherein said injecting comprises introducing the vector into the muscle through a needle.

9. The method of claim 1, wherein said injecting involves introducing the vector into muscle by biolistic delivery.

10. The method of claim 1, wherein said at least one vector is at least two vectors and wherein each of said polypeptide chains is encoded by a separate vector.

11. The method of claim 1, wherein said method further comprises the step of positioning electrodes near said injection site such that current travelling through the electrodes travels through the injection site and transiently increases muscle membrane permeability.

12. The method of claim 11, wherein said transient increase in muscle membrane permeability is achieved with an electrical current having a field strength in the range of from about 25 V/cm to less than 300 V/cm.

13. The method of claim 1, wherein said muscle is skeletal muscle.

14. The method of claim 1, wherein said protein comprises one or more antigenic determinants foreign to the individual, thereby generating an immune response to the expressed protein in the individual.

15. The method of claim 14, wherein said immune response includes the production of antibodies in the serum of the individual to the one or more of the foreign antigenic determinants of the protein.

16. A method of obtaining antibodies to a protein that comprises at least two different polypeptide chains, said method comprising injecting into muscle of an individual at least one expression vector that encodes the polypeptide chains, such that uptake of the vector into muscle cells results in secretion of the protein, and obtaining the antibodies from the individual, wherein said protein comprises one or more antigenic determinants foreign to the individual.

17. The method of claim 16, wherein said antibodies are obtained by obtaining a source of fluid from the individual.

18. The method of claim 17, wherein said fluid is serum.

19. The method of claim 16, wherein said protein is an immunoglobulin.

20. The method of claim 16, wherein said individual is a human.

21. The method of claim 16, wherein said at least one vector is at least two vectors and wherein each of said polypeptide chains is encoded by a separate vector.

22. The method of claim 16, wherein said method further comprises the step of positioning electrodes near said injection site such that current travelling through the electrodes travels through the injection site and transiently increases muscle membrane permeability.

23. The method of claim 22, wherein said electrically stimulating the muscle is achieved with an electrical current having a field strength in the range of from about 25 V/cm to less than 300 V/cm.

24. The method of claim 16, wherein said muscle is skeletal muscle.

25. A method of immunizing an individual, comprising injecting at least one expression vector into the muscle of the individual, said vector encoding the light chains and the heavy chains of a bispecific antibody, said bispecific antibody having a first binding site specific for a cell surface marker of an antigen presenting cell of the individual and a second binding site specific for an antigen to which immunization is desired, wherein uptake of the vector into muscle cells results in secretion of the bispecific antibody in the circulation of the individual, and administering the antigen to the individual so that the antigen is targeted to antigen presenting cells by the bispecific antibody.

26. The method of claim 25, wherein said cell surface marker is selected from the group consisting of MHC class II molecule, B7 molecule, IgD, Fc-receptor, CD40, and Toll receptor.

27. The method of claim 25, wherein said bispecific antibody is made up of separate heavy and light chains.

28. The method of claim 25, wherein said bispecific antibody is a single polypeptide.

29. The method of claim 25, wherein said second binding site is specific for a peptide sequence and wherein said antigen is engineered to contain the polypeptide sequence.

30. The method of claim 25, wherein said at least one vector is two vectors and wherein of said heavy chain and said light chain are encoded by a separate vector.

31. The method of claim 25, wherein said antigen is administered by recombinantly expressing the antigen in the individual.

32. The method of claim 25, wherein said vector codes for each of a full-length light chain and a full-length heavy chain.

33. The method of claim 25, wherein said individual is a human.

34. The method of claim 25, wherein at least one chain of said bispecific antibody has constant region sequence from human immunoglobulin.

35. The method of claim 25, wherein at least one chain of said immunoglobulin has variable region sequence from human immunoglobulin.

36. The method of claim 25, wherein said method further comprises the step of positioning electrodes near said injection site such that current travelling through the electrodes travels through the injection site and transiently increases muscle membrane permeability.

37. The method of claim 36, wherein said transient increase in muscle membrane permeability is achieved with an electrical current having a field strength in the range of from about 25 V/cm to less than 300 V/cm.

38. The method of claim 25, wherein said muscle is skeletal muscle.

39. A method of immunizing an individual, comprising injecting at least one expression vector into the muscle of the individual, said vector comprising nucleic acid encoding an antibody fusion protein, said fusion protein comprising an antibody specific for a cell surface marker of an antigen presenting cell of the individual, said antibody fused to a polypeptide antigen to which immunization is desired, wherein uptake of the vector into muscle cells results in secretion of the antibody fusion protein, said secreted fusion protein functioning to target the antigen to the surface of antigen presenting cells of the individual.

40. The method of claim 39, wherein said cell surface marker is selected from the group consisting of MHC class II molecule, B7 molecule, IgD, Fc-receptor, CD40, and Toll receptor.

41. The method of claim 39, wherein said antibody comprises separate heavy and light chains.

42. The method of claim 39, wherein said antibody comprises heavy and light chains as a single polypeptide.

43. The method of claim 39, wherein said antigen is fused to the heavy chain of the antibody.

44. The method of claim 39, wherein said antigen is fused to the light chain of the antibody.

45. The method of claim 39, wherein said vector codes for each of a full-length light chain and a full-length heavy chain.

46. The method of claim 39, wherein said individual is a human.

47. The method of claim 39, wherein at least one chain of said immunoglobulin has constant region sequence from human.

48. The method of claim 39, wherein at least one chain of said immunoglobulin has variable region sequence from human.

49. The method of claim 39, wherein said at least one vector is two vectors and wherein of said heavy chain and said light chain are encoded by a separate vector.

50. The method of claim 39, wherein said method further comprises the step of positioning electrodes near said injection site such that current travelling through the electrodes travels through the injection site and transiently increases muscle membrane permeability.

51. The method of claim 50, wherein said transient increase in muscle membrane permeability is achieved with an electrical current having a field strength in the range of from about 25 V/cm to less than 300 V/cm.

52. A method of testing whether a protein comprising at least two different polypeptide chains is biologically active when expressed, said method comprising, injecting into muscle of the individual at least one expression vector that encodes the two polypeptide chains, such that uptake of the vector into muscle cells results in production of the polypeptide chains and secretion of the protein, and evaluating at least one biological activity of said protein.

53. The method of claim 52, wherein said biological activity occurs in the individual.

54. The method of claim 52, wherein a source of said protein is obtained from the individual and biological activity determined.

55. The method of claim 52, wherein said protein is an antibody.

56. The method of claim 55, wherein said biological activity is antigen specificity.

57. The method of claim 55, wherein said vector codes for each of a full-length light chain and a full-length heavy chain.

58. The method of claim 52, wherein said at least one vector is two vectors and wherein each polypeptide is encoded by a separate vector.

59. The method of claim 55, wherein said nucleic acid encoding the polypeptides has been mutated.

60. The method of claim 52, wherein said method further comprises the step of positioning electrodes near said injection site such that current travelling through the electrodes travels through the injection site and transiently increases muscle membrane permeability.

61. The method of claim 60, wherein said transient increase in muscle membrane permeability is achieved with an electrical current having a field strength in the range of from about 25 V/cm to less than 300 V/cm.

62. The method of claim 2, wherein said immunoglobulin is mouse immunoglobulin.

63. The method of claim 19, wherein said immunoglobulin is mouse immunoglobulin.

64. The method of claim 25, wherein said antibody is mouse antibody.

65. The method of claim 39, wherein said antibody is mouse antibody.

66. The method of claim 55, wherein said antibody is mouse antibody.


SEQUENCE LISTING (4 sequences)

SEQ ID NO: 1 - DNA, 35 bp, Artificial Sequence
  Description of Artificial Sequence Primer
ggtgtgcattccgacattgttctgacacagtctcc

SEQ ID NO: 2 - DNA, 39 bp, Artificial Sequence
  Description of Artificial Sequence Primer
acgtacgttctactcacgcttgatttccagcttggtgcc

SEQ ID NO: 3 - DNA, 23 bp, Artificial Sequence
  Description of Artificial Sequence Primer
caggtccaattgcagcagtctgg

SEQ ID NO: 4 - DNA, 37 bp, Artificial Sequence
  Description of Artificial Sequence Primer
gacgtacgactcacctgaggagaccgtgactgaggtt